(19) United States
(12) Patent Application Publication
(10) Pub. No.: US 2013/0023635 A1
(43) Pub. Date: Jan. 24, 2013

(54) CATALYSTS BASED ON HETEROCYCLIC-8-ANILINOQUINOLINE LIGANDS

(71) Applicants: Ilya E. NIFANT'EV, Moscow (RU); Sander Nagy, Naperville, IL (US); Vladimir V. Bagrov, Moscow (RU); Pavel V. Ivchenko, Moscow (RU); Shahram Mihan, Bad Soden (DE)

(72) Inventors: Ilya E. NIFANT'EV, Moscow (RU); Sander Nagy, Naperville, IL (US); Vladimir V. Bagrov, Moscow (RU); Pavel V. Ivchenko, Moscow (RU); Shahram Mihan, Bad Soden (DE)

(21) Appl. No.: 13/185,128

(22) Filed: Jul. 18, 2011

(51) Int. Cl. B01J 31/12; C07F 15/00; C07D 409/04; C08F 4/64; C07D 401/04

(52) U.S. Cl. 526/170; 502/155; 502/103; 546/167; 546/10

(57) ABSTRACT

A catalyst system useful for polymerizing olefins is disclosed. The catalyst system comprises an activator and a Group 4 metal complex. The complex incorporates a dianionic, tridentate heterocyclic-8-anilinoquinoline ligand. In one aspect, a supported catalyst system is prepared by first combining a boron compound having Lewis acidity with excess alumoxane to produce an activator mixture, followed by combining the activator mixture with a support and the dianionic, tridentate Group 4 metal complex. The Group 4 metal complexes are easy to synthesize, support, and activate, and they enable facile production of high-molecular-weight polyolefins.

### FIELD OF THE INVENTION

The invention relates to non-metallocene catalyst systems useful for polymerizing olefins. The catalyst systems incorporate a tridentate dianionic ligand.

### BACKGROUND OF THE INVENTION

While Ziegler-Natta catalysts are a mainstay for polyolefin manufacture, single-site (metallocene and non-metallocene) catalysts represent the industry's future. These catalysts are often more reactive than Ziegler-Natta catalysts, and they produce polymers with improved physical properties. The improved properties include controlled molecular weight distribution, reduced low molecular weight extractables, enhanced incorporation of □-olefin comonomers, lower polymer density, controlled content and distribution of long-chain branching, and modified melt rheology and relaxation characteristics.

Traditional metallocenes incorporate one or more cyclopentadienyl (Cp) or Cp-like anionic ligands such as indenyl, fluorenyl, or the like, that donate pi-electrons to the transition metal. Non-metallocene single-site catalysts, including ones that capitalize on the chelate effect, have evolved more recently. Examples are the bidentate 8-quinolinoxy or 2-pyridinoxy complexes of Nagy et al. (see U.S. Pat. No. 5,637,660), the late transition metal bisimines of Brookhart et al. (see Chem. Rev. 100 (2000) 1169), and the diethylenetriamine-based tridentate complexes of McConville et al. or Shrock et al. (e.g., U.S. Pat. Nos. 5,889,128 and 6,271,323).

In numerous recent examples, the bi- or tridentate complex incorporates a pyridyl ligand that bears a heteroatom β- or γ- to the 2-position of the pyridine ring. This heteroatom, typically nitrogen or oxygen, and the pyridyl nitrogen chelate the metal to form a five- or six-membered ring. For some examples, see U.S. Pat. Nos. 7,439,205; 7,423,101; 7,157,400; 6,653,417; and 6,103,657 and U.S. Pat. Appl. Publ. No. 2008/0177020. In some of these complexes, an aryl substituent at the 6-position of the pyridine ring is also available to interact with the metal through C—H activation to form a tridentate complex (see, e.g., U.S. Pat. Nos. 7,115,689; 6,953,764; 6,706,829). Unfortunately, some of these complexes are tricky to prepare, and they are most useful unsupported; our own attempts to prepare similar complexes and support them on silica, for example, met with mixed results.

Less frequently, quinoline-based bi- or tridentate complexes have been described (see, e.g., U.S. Pat. Nos. 7,253,133; 7,049,378; 6,939,969; 6,103,657; 5,637,660 and Organometallics 16 (1997) 3282). The quinoline complexes disclosed in the art lack an 8-anilino substituent, a 2-aryl substituent, or both, and/or they are not dianionic and tridentate.

WO 2011/011041 describes catalyst systems based on 2-aryl-8-anilinquinoline ligands, however this document does not suggest that the aryl substituent can be replaced with an heterocyclic radical.

New non-metallocene catalyst system useful for making polyolefins continue to be of interest. In particular, tridentate complexes that can be readily synthesized from inexpensive reagents are needed. The complexes should not be useful only in homogeneous environments; a practical complex can be supported on silica and readily activated toward olefin polymerization with alumoxanes or boron-containing cocatalysts. Ideally, the catalysts have the potential to make ethylene copolymers having high or very high molecular weights and can be utilized in high-temperature solution polymerizations.

### SUMMARY OF THE INVENTION

The invention relates to catalyst system useful for polymerizing olefins. The catalyst system comprises an activator and a Group 4 metal complex. The complex incorporates a dianionic, tridentate heterocyclic-8-anilinoquinoline ligand. In one aspect, a supported catalyst system is prepared by first combining a boron compound having Lewis acidity with excess alumoxane to produce an activator mixture, followed by combining the activator mixture with a support and the dianionic, tridentate Group 4 metal complex. The Group 4 metal complex are easy to synthesize, support, and activate, and they enable facile production of high-molecular-weight polyolefins.

### DETAILED DESCRIPTION OF THE INVENTION

The catalysts system of the invention comprises:

A) a Group 4 transition metal complex having formula (I)

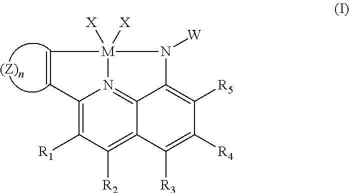

B) one or more activators;
Wherein in the compound of formula (I):
   M is a metal selected from the group comprising zirconium, titanium, and hafnium; zirconium and titanium are particularly preferred; more particularly zirconium is preferred;
   X, equal to or different from each other, is a halogen atom, a R, OR, SR, NR₂ or PR₂ group wherein R is a linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radical; or two X groups can be joined together to form a divalent R′ group wherein R′ is a C₁-C₂₀-alkylidene, C₆-C₂₀-arylidene, C₇-C₂₀-alkylarylidene, or C₇-C₂₀-arylalkylidene divalent radical optionally containing heteroatoms belonging to groups 13-17 of the Periodic Table of the Elements; preferably X is a halogen atom or R group; more preferably X is a C₇-C₄₀-alkylaryl radical such as benzyl radical;
   R¹, R², R³, R⁴ and R⁵, equal to or different from each other, are hydrogen atoms or C₁-C₄₀ hydrocarbon groups optionally containing one or more heteroatoms belonging to groups 13-17 of the Periodic Table of the Elements; preferably R¹, R², R³, R⁴ and R⁵ equal to or different from each other, are hydrogen atoms or linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radicals, optionally containing one or more heteroatoms belonging to groups 13-17 of the Periodic Table of the Elements; more preferably R¹, R², R³, R⁴ and R⁵ are hydrogen atoms;
   Z is a divalent radical selected from: CR⁶, S, O, NR⁶, PR⁶, N, P provided that at least one Z is different from CR⁶, preferably provided that only one Z is different from CR⁶; and preferably the ring formed by Z is aromatic;
   wherein R⁶ equal to or different from each other, are hydrogen atoms or C₁-C₄₀ hydrocarbon groups optionally containing one or more heteroatoms belonging to groups 13-17 of the Periodic Table of the Elements; preferably R⁶, equal to or different from each other, are hydrogen atoms or linear or branched, cyclic or acyclic, C₁-C₄-alkyl, C₂-C₄₀ alkenyl, C₂-C₄₀ alkynyl, C₆-C₄₀-aryl, C₇-C₄₀-alkylaryl or C₇-C₄₀-arylalkyl radicals, optionally containing one or more heteroatoms belonging to groups 13-17 of the Periodic Table of the Elements; or two R⁶ can be joined to form one or more C₃-C₂₀ membered ring that can be aliphatic or aromatic and one or more carbon atoms can be optionally substituted with heteroatoms belonging to groups 13-17 of the Periodic Table of the Elements, and can have on its turn C₁-C₄₀ hydrocarbon substituents optionally containing heteroatoms belonging to groups 13-17 of the Periodic Table of the Elements; preferably R⁶ equal to or different from each other, are hydrogen atoms or C₁-C₄₀-alkyl, C₆-C₄₀-aryl radicals;
   n ranges from 3 to 4; preferably n is 3;
   W is a C₆-C₄₀-aryl aryl radical that can be substituted with one or more, G group wherein
   G, equal to or different from each other, are linear or branched C₁-C₄₀-alkyl, C₂-C₄₀ alkenyl, C₂-C₄₀ alkynyl radicals; preferably W is a phenyl radical substituted in position 2 and 6 by G groups, more preferably W is a phenyl radical substituted in positions 2 and 6 with linear or branched C₁-C₄₀-alkyl radicals; preferred alkyl radicals are methyl, ethyl, propyl, isopropyl, tert-butyl radicals.

The catalyst system includes one or more activators. The activator helps to ionize the complex and activate the catalyst. Suitable activators are well known in the art. Examples include alumoxanes (methyl alumoxane (MAO), PMAO, ethyl alumoxane, diisobutyl alumoxane), alkylaluminum compounds (triethylaluminum, diethylaluminum chloride, trimethylaluminum, triisobutylaluminum), and the like. Suitable activators include boron and aluminum compounds having Lewis acidity such as ionic borates or aluminates, organoboranes, organoboronic acids, organoborinic acids, and the like. Specific examples include lithium tetrakis(pentafluorophenyl)borate, lithium tetrakis(pentafluorophenyl)aluminate, anilinium tetrakis(pentafluorophenyl)-borate, trityl tetrakis(pentafluorophenyl)borate (“F20”), tris(pentafluorophenyl)-borane (“F15”), triphenylborane, tri-n-octylborane, bis(pentafluorophenyl)borinic acid, pentafluorophenylboronic acid, and the like. These and other suitable boron-containing activators are described in U.S. Pat. Nos. 5,153,157, 5,198,401, and 5,241,025, the teachings of which are incorporated herein by reference. Suitable activators also include aluminoboronates—reaction products of alkyl aluminum compounds and organoboronic acids—as described in U.S. Pat. Nos. 5,414,180 and 5,648,440, the teachings of which are incorporated herein by reference. Particularly preferred activators are alumoxanes, boron compounds having Lewis acidity, and mixtures thereof.

Preferably the compound of formula (I) have formulas (Ia) or (Ib)

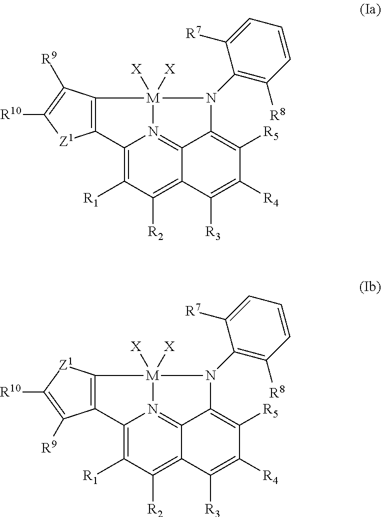

Wherein M, X, R¹, R², R³, R⁴ and R⁵ have been described above;

R⁷ and R⁸, equal to or different from each other, are C₁-C₄₀ hydrocarbon groups optionally containing one or more heteroatoms belonging to groups 13-17 of the Periodic Table of the Elements; preferably R⁷ and R⁸, equal to or different from each other, are linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radicals, optionally containing one or more heteroatoms belonging to groups 13-17 of the Periodic Table of the Elements; more preferably R⁷ and R⁸ are linear or branched C₁-C₁₀-alkyl radicals, such as methyl, ethyl, n-propyl, isopropyl, n-butyl, and tert-butyl;

R⁹ and R¹⁰, equal to or different from each other, are hydrogen atoms or C₁-C₄₀ hydrocarbon groups optionally containing one or more heteroatoms belonging to groups 13-17 of the Periodic Table of the Elements or R⁹ and R¹⁰ can be joined to form a C₅-C₆ membered ring; preferably a phenyl ring; preferably R⁹ and R¹⁰, equal to or different from each other, are linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radicals, optionally containing one or more heteroatoms belonging to groups 13-17 of the Periodic Table of the Elements or R⁹ and R¹⁰ can be joined to form a phenyl ring; preferably; even more preferably R¹⁰ is a C₁-C₁₀-alkyl radical or is joined with R⁹ to form a phenyl ring; even more preferably R⁹ is a hydrogen atom or is joined with R¹⁰ to form a phenyl ring. Z¹ is S, O, or NR⁶ wherein R⁶ has been described above; preferably Z is S or NR⁶ wherein R⁶ is a C₁-C₁₀-alkyl or a C₆-C₂₀-aryl radical; more preferably R⁶ is a phenyl radical or a methyl, ethyl, n-propyl, isopropyl, n-butyl, and tert-butyl radical;

The catalyst system of the present invention can further comprise an inert support c). Preferably inert support are inorganic oxide such as silica, alumina, silica-alumina, magnesia, titania, zirconia, clays, zeolites, or the like. Silica is preferred. When silica is used, it preferably has a surface area in the range of 10 to 1000 m²/g, more preferably from 50 to 800 m²/g and most preferably from 200 to 700 m²/g. Preferably, the pore volume of the silica is in the range of 0.05 to 4.0 mL/g, more preferably from 0.08 to 3.5 mL/g, and most preferably from 0.1 to 3.0 mL/g. Preferably, the average particle size of the silica is in the range of 1 to 500 microns, more preferably from 2 to 200 microns, and most preferably from 2 to 45 microns. The average pore diameter is typically in the range of 5 to 1000 angstroms, preferably 10 to 500 angstroms, and most preferably 20 to 350 angstroms.

The support is preferably treated thermally, chemically, or both prior to use by methods well known in the art to reduce the concentration of surface hydroxyl groups. Thermal treatment consists of heating (or “calcining”) the support in a dry atmosphere at elevated temperature, preferably greater than 100° C., and more preferably from 150 to 800° C., prior to use. A variety of different chemical treatments can be used, including reaction with organo-aluminum, -magnesium, -silicon, or -boron compounds. See, for example, the techniques described in U.S. Pat. No. 6,211,311, the teachings of which are incorporated herein by reference.

Highly active non-metallocene catalysts of the invention can be made by using a particular sequence for activating and supporting the tridentate dianionic complexes. One method of preparing a supported catalyst useful for polymerizing olefins comprises two steps. In a first step, a boron compound having Lewis acidity (as described earlier) is combined with excess alumoxane, preferably methylalumoxane, to produce an activator mixture. In a second step, the resulting activator mixture is combined with a support, preferably silica, and a complex which comprises a Group 4 transition metal and a dianionic, tridentate 2-aryl-8-anilinoquinoline ligand. In one approach, the activator mixture is combined with the complex first, followed by the support. However, the order can be reversed; thus, the activator mixture can be combined with the support first, followed by the complex.

In a typical example, the boron compound is combined with excess MAO in a minimal amount of a hydrocarbon. The complex is added and the combined mixture is then added to a large proportion of calcined silica in an incipient wetness technique to provide the supported catalyst as a free-flowing powder.

A further object of the present invention is the organic ligand of formula (II)

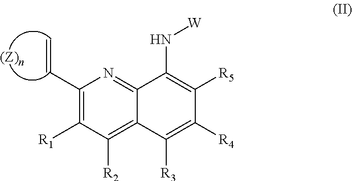

Wherein Z, n, R¹, R², R³, R⁴, R⁵ and W have been described above.

Preferably the ligand of formula (II) has formula (IIa) or (IIb)

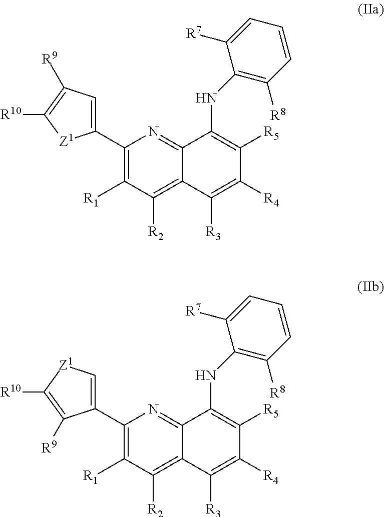

Wherein Z¹, R¹, R², R³, R⁴, R⁵, R⁷, R⁸, R⁹ and R¹⁰ have been described above.

With the catalyst system of the present invention it is possible to polymerize alpha-olefins in high yield to obtain polymers having high molecular weight. Thus a further object of the present invention is a process for polymerizing one or more alpha olefins of formula CH₂═CHT wherein T is hydrogen or a C₁-C₂₀ alkyl radical comprising the step of contacting said alpha-olefins of formula CH₂═CHT under polymerization conditions in the presence of the catalyst system described above.

Preferred α-olefins are ethylene, propylene, 1-butene, 1-hexene, 1-octene.

The catalyst system of the present invention is particularly fit for the polymerization of ethylene or copolymerization of ethylene and propylene, 1-butene, 1-hexene and 1-octene. Thus a further object of the present invention is a process for polymerizing ethylene and optionally one or more alpha olefins selected from propylene, 1-butene, 1-hexene and 1-octene comprising the step of contacting ethylene and optionally said alpha-olefins under polymerization conditions in the presence of the catalyst system described above.

Many types of olefin polymerization processes can be used. Preferably, the process is practiced in the liquid phase, which can include slurry, solution, suspension, or bulk processes, or a combination of these. High-pressure fluid phase or gas phase techniques can also be used. In a preferred olefin polymerization process, a supported catalyst of the invention is used. The polymerizations can be performed over a wide temperature range, such as −30° C. to 280° C. A more preferred range is from 30° C. to 180° C.; most preferred is the range from 60° C. to 100° C. Olefin partial pressures normally range from 15 psig to 50,000 psig. More preferred is the range from 15 psig to 1000 psig.

The invention includes a high-temperature solution polymerization process. By “high-temperature,” we mean at a temperature normally used for solution polymerizations, i.e., preferably greater than 130° C., and most preferably within the range of 135° C. to 250° C.

The following examples merely illustrate the invention. Those skilled in the art will recognize many variations that are within the spirit of the invention and scope of the claims.

### EXAMPLES

All intermediate compounds and complexes synthesized give satisfactory ¹H NMR spectra consistent with the structures indicated.

### Synthesis of Dibenzylzirconium N-(2,6-diisopropylphenyl)-2-(5-methyl-3-thienyl)-8-quinolinamide (A-1)

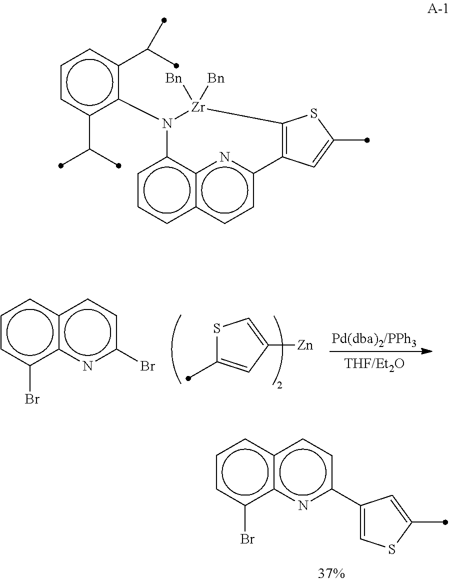

### 8-Bromo-2-(5-methyl-3-thienyl)quinoline

To a solution of 2-methyl-4-bromothiphene (1.77 g, 10 mmol) in 20 ml of ether a BuLi solution in hexane (10 mmol) was added at −50° C. After stirring for 2 h at this temperature, a solution of ZnCl2 (0.68 g, 5 mmol) in 10 ml of THF was added, allowed to reach ambient temperature and continued the reaction for an additional 20 min. The catalytic system Pd(dba)₂ (100 mg) and PPh₃ (100 mg) was added to the resulting solution, followed in 5 minutes by 2,8-dibromoquinoline (2.87, 10 mmol). The reaction mixture was stirred overnight, treated with a 10% solution of NH4Cl, the organic phase separated, while the aqueous layer extracted with ether. The combined organic phases were dried over MgSO4, evaporated and the residue purified on a silica column using a benzene-hexane (1:4) eluent, resulting in 1.12 g of product (37%).

NMR ¹H (CDCl₃): 8.08 (d, 1H), 8.03 (d, 1H); 7.86 (s, 1H); 7.74 (d, 1H); 7.68 (br.s., 1H); 7.31 (t, 1H); 2.59 (s, 3H).

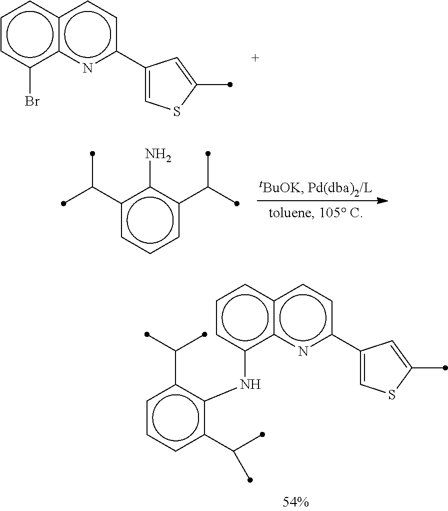

### N-(2,6-diisopropylphenyl)-2-(5-methyl-3-thienyl)-8-quinolinamine

A mixture of the 8-bromo-2-(5-methyl-3-thienyl)quinoline (3.7 mmol), 2,6-diisopropylaniline (0.71 g, 4.0 mmol), sodium tert-butylate (0.5 g), Pd(dba)₂ (40 mg) and (N-[2′-(dicyclohexylphosphino)[1,1′-biphenyl]-2-yl]-N,N-dimethylamine) (60 mg) in 8 ml of toluene reacted under argon at 105° C. for 12 h. After addition of water, the organic phase separated and the aqueous layer extracted with ether. The combined organic phases were dried over MgSO4, evaporated and the residue purified on a silica column using a petroleum ether-benzene (5:1) eluent, resulting in 0.8 g of product (54%).

NMR ¹H (CDCl₃): 8.14 (d, 1H); 7.80 (m, 2H); 7.75 (br.s., 1H); 7.61 (br.s., 1H); 7.34 (m, 4H); 7.24 (t, 1H); 7.09 (d, 1H); 6.33 (d, 1H); 3.30 (m, 2H); 2.62 (s, 3H); 1.27 (d, 6H); 1.19 (d, 6H).

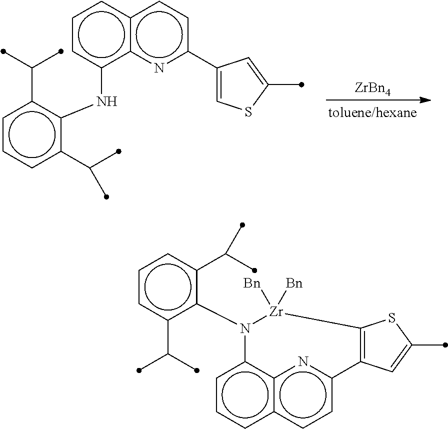

### Dibenzylzirconium N-(2,6-diisopropylphenyl)-2-(5-methyl-3-thienyl)-8-quinolinamide (A-1)

Solution of tetrabenzylzirconium (0.87 g, 1.92 mmol) in toluene-hexane (1:1, 10 ml) was added at 0° C. to a solution of N-(2,6-diisopropylphenyl)-2-(5-methyl-3-thienyl)-8-quinolinamine (0.64 g, 1.60 mmol) in toluene-hexane (1:1, 15 ml). The mixture was allowed to warm to room temperature, stirred overnight, and evaporated. The residue was recrystallized from hexane with the yield of 0.65 g (60%).

NMR ¹H(C₆D₆) □: 7.60 (d, 1H); 7.41-6.62 (groups of m, 17H); 6.27 (d, 1H); 3.44 (m, 2H); 2.53 (d, 2H); 2.33 (s, 3H); 1.65 (d, 2H); 1.23 (d, 6H); 1.01 (d, 6H).

### Synthesis of Dibenzylzirconium N-(2,6-diisopropylphenyl)-2-(5-methyl-2-thienyl)-8-quinolinamide (A-2)

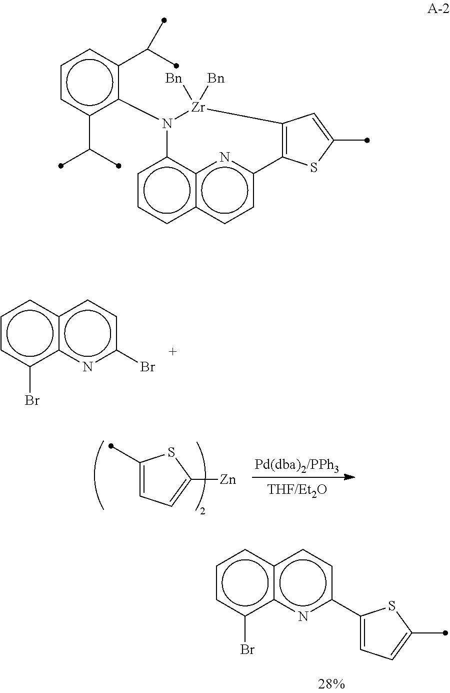

### 8-Bromo-2-(5-methyl-2-thienyl)quinoline

To a solution of 2-methyl-5-bromothiphene (1.77 g, 10 mmol) in 20 ml of ether a BuLi solution in hexane (10 mmol) was added at −50° C. After stirring for 2 h at this temperature, a solution of ZnCl2 (0.68 g, 5 mmol) in 10 ml of THF was added, allowed to reach ambient temperature and continued the reaction for an additional 20 min. The catalytic system Pd(dba)₂ (100 mg) and PPh₃ (100 mg) was added to the resulting solution, followed in 5 minutes by 2,8-dibromoquinoline (2.87, 10 mmol). The reaction mixture was stirred overnight, treated with a 10% solution of NH4Cl, the organic phase separated, while the aqueous layer extracted with ether. The combined organic phases were dried over MgSO4, evaporated and the residue purified on a silica column using a benzene-hexane (1:4) eluent, resulting in 0.85 g of product (28%).

NMR ¹H (CDCl₃): 8.10 (d, 1H), 8.05 (d, 1H); 7.77 (d, 1H); 7.74 (d, 1H); 7.66 (d, 1H); 7.33 (t, 1H); 2.57 (s, 3H).

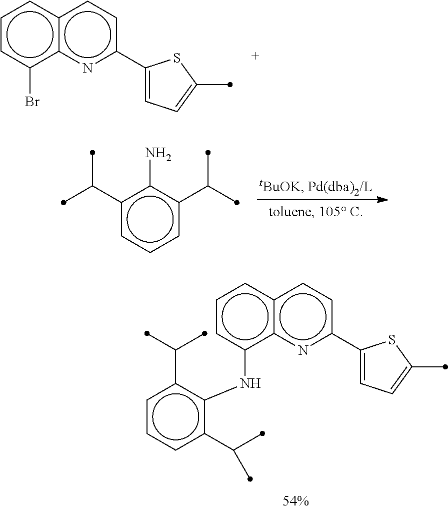

### N-(2,6-diisopropylphenyl)-2-(5-methyl-2-thienyl)-8-quinolinamine

A mixture of the 8-bromo-2-(5-methyl-2-thienyl)quinoline (2.8 mmol), 2,6-diisopropylaniline (0.71 g, 4.0 mmol), sodium tert-butylate (0.5 g), Pd(dba)₂ (40 mg) and (N-[2′-(dicyclohexylphosphino)[1,1′-biphenyl]-2-yl]-N,N-dimethylamine) (60 mg) in 8 ml of toluene reacted under argon at 105° C. for 12 h. After addition of water, the organic phase separated and the aqueous layer extracted with ether. The combined organic phases were dried over MgSO4, evaporated and the residue purified on a silica column using a petroleum ether-benzene (5:1) eluent, resulting in 0.8 g of product (54%).

NMR ¹H (CDCl₃): 8.13 (d, 1H); 7.77 (m, 2H); 7.72 (br.s., 1H); 7.58 (br.s., 1H); 7.31 (m, 4H); 7.21 (t, 1H); 7.06 (d, 1H); 6.29 (d, 1H); 3.27 (m, 2H); 2.59 (s, 3H); 1.24 (d, 6H); 1.16 (d, 6H).

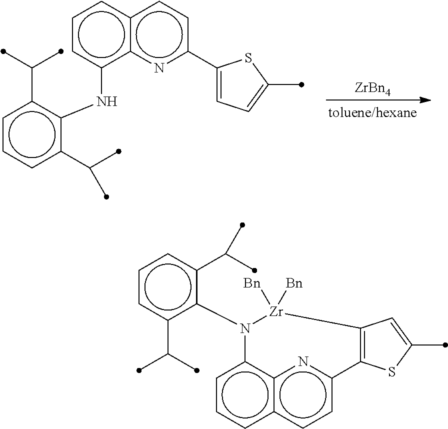

### Dibenzylzirconium N-(2,6-diisopropylphenyl)-2-(5-methyl-2-thienyl)-8-quinolinamide (A-2)

Solution of tetrabenzylzirconium (0.37 g, 0.81 mmol) in toluene-hexane (1:1, 3 ml) was added at 0° C. to a solution of N-(2,6-diisopropylphenyl)-2-(5-methyl-2-thienyl)-8-quinolinamine (0.27 g, 0.67 mmol) in toluene-hexane (1:1, 7 ml). The mixture was allowed to warm to room temperature, stirred overnight, and evaporated. The residue was recrystallized from hexane. The yield 0.21 g (46%).

NMR ¹H (C₆D₆) □: 7.58 (d, 1H); 7.28-6.47 (groups of m, 17H); 6.19 (d, 1H); 3.52 (m, 2H); 2.59 (d, 2H); 2.28 (s, 3H); 1.77 (d, 2H); 1.26 (d, 6H); 1.03 (d, 6H).

### Synthesis of Dibenzylzirconium N-(2,6-dimethylphenyl)-2-(1-phenyl-1H-indol-2-yl)-8-quinolinamide (A-3)

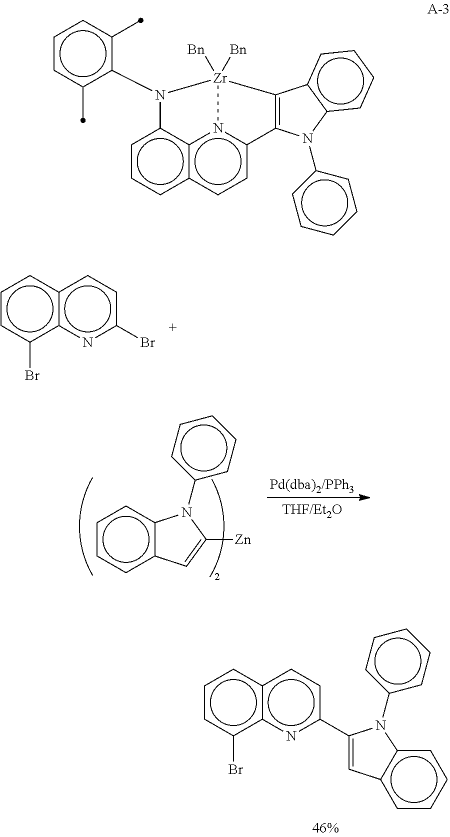

### 8-Bromo-2-(1-phenyl-1H-indol-2-yl)quinoline

To a solution of N-phenylindole (3.86 g, 20 mmol) in 30 ml of THF a BuLi solution in hexane (20 mmol) was added at 0° C. After stirring for 2 h at ambient temperature the mixture was cooled again to 0° C. and a solution of ZnCl2 (1.36 g, 10 mmol) in 20 ml of THF was added, allowed to reach ambient temperature and continued the reaction for an additional 20 min. The catalytic system Pd(dba)₂ (200 mg) and PPh₃ (200 mg) was added to the resulting solution, followed in 5 minutes by 2,8-dibromoquinoline (4.9 g, 17 mmol). The reaction mixture was stirred overnight, treated with a 10% solution of NH4Cl, the organic phase separated, while the aqueous layer extracted with ether. The combined organic phases were dried over MgSO4, evaporated and the residue washed with ethanol, resulting in 3.6 g of product (45.5%).

NMR ¹H (CDCl₃): 7.99 (d, 1H); 7.93 (d, 1H); 7.79 (d, 1H); 7.65 (t, 2H); 7.47 (m, 2H), 7.42 (m, 4H); 7.28 (m, 4H).

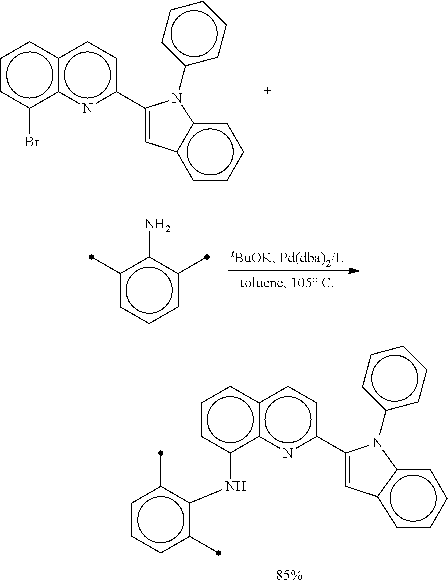

### N-(2,6-dimethylphenyl)-2-(1-phenyl-1H-indol-2-yl)-8-quinolinamine

A mixture of the 8-bromo-2-(1-phenyl-1H-indol-2-yl)quinoline (1.6 g, 4 mmol), 2,6-diisopropylaniline (0.71 g, 4.0 mmol), sodium tert-butylate (0.75 g), Pd(dba)₂ (60 mg) and (N-[2′-(dicyclohexylphosphino)[1,1′-biphenyl]-2-yl]-N,N-dimethylamine) (90 mg) in 12 ml of toluene reacted under argon at 105° C. for 12 h. After addition of water, the organic phase separated and the aqueous layer extracted with ether. The combined organic phases were dried over MgSO4, evaporated and the residue was washed with ethanol, resulting in 1.5 g of product (85%).

NMR ¹H (CDCl₃): 8.07 (d, 1H); 7.94 (d 1H); 7.82 (m, 1H); 7.48 (d, 2H); 7.40 (m, 3H); 7.27 (m, 2H); 7.18 (m, 6H); 7.02 (d, 1H); 6.17 (br.s., 1H); 6.10 (d, 1H); 2.14 (s, 6H).

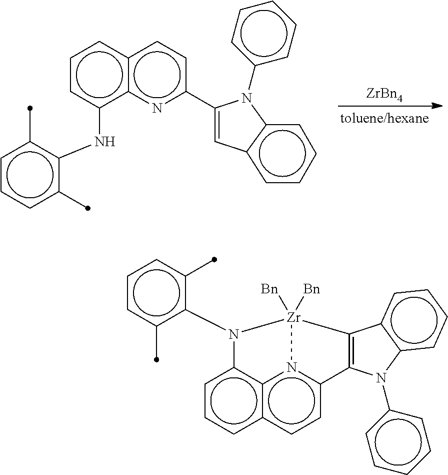

### Dibenzylzirconium N-(2,6-dimethylphenyl)-2-(1-phenyl-1H-indol-2-yl)-8-quinolinamide (A-3)

Solution of tetrabenzylzirconium (0.45 g, 0.99 mmol) in toluene-hexane (1:1, 10 ml) was added at −20° C. to a solution of N-(2,6-dimethylphenyl)-2-(1-phenyl-1H-indol-2-yl)-8-quinolinamine (0.31 g, 0.71 mmol) in toluene-hexane (1:1, 10 ml). The mixture was allowed to warm to room temperature, stirred overnight, and evaporated. The residue was recrystallized from hexane. The yield 0.22 g (44%). Yellow crystalline powder.

NMR ¹H (C₆D₆) □: 8.30 (d, 1H); 7.35 (t, 1H); 7.21 (t, 1H); 7.19-6.63 (groups of m, 22H); 6.46 (d, 1H); 6.23 (d, 1H); 2.57 (d, 2H); 2.20 (d, 2H); 2.15 (s, 6H).

### Preparation of Supported Catalysts

Trityl tetrakis(pentafluorophenyl)borate (“F20, 0.03 g”) is added to methylalumoxane (30 wt. % solution of MAO in toluene, 1.4 mL), and the mixture is stirred for 15 min. A specified amount of complex precursor indicated in table 1 is added to the MAO/borate solution, and the mixture stirs for an additional 15 min. The resulting product is slowly added to a stirred bed of silica (Davison 948, calcined at 600° C. for 6 h, 1.0 g). The resulting free-flowing powder is used in polymerization tests.

### Ethylene Polymerization: General Procedure

In a representative procedure a dry, 2-L stainless-steel autoclave is charged with isobutane (1.0 L), triisobutylaluminum (1 M solution in hexanes, 2 mL), 1-butene (100 mL) and, optionally, hydrogen, and the contents are heated to 70° C. and pressurized with ethylene (22.5 psi partial pressure). Polymerization is started by injecting the catalyst with a small quantity of isobutane. The temperature is maintained at 70° C., and ethylene is supplied on demand throughout the test. The reaction is terminated after an hour by cooling the reactor and venting its contents. The results of the polymerization tests are reported on table 1.

TABLE 1

| | | Ex | | | |
|---|---|---|---|---|---|
| | | 1 | 2 | 3 | 4 |
| Comp | | A-1 (19) | A-1 (19) | A-2 (19) | A-3 (21) |
| (mg) | | | | | |
| Hydrogen | (cm³) | 0 | 7 | 0 | 0 |
| Activity | Kg pol/mol | 6207 | 5014 | 4842 | 1265 |
| | compound/h | | | | |

The catalyst system of the present invention shows a good activity in the copolymerization of ethylene

We claim:

1. A catalyst system for the polymerization of alpha olefins comprising:
A) a Group 4 transition metal complex having formula (I) 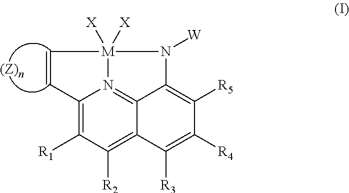
and
B) one or more activators;
wherein in the compound of formula (I):
M is a metal selected from the group comprising zirconium, titanium, and hafnium;
X, equal to or different from each other, is a halogen atom, a R, OR, SR, NR₂ or PR₂ group wherein R is a linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radical; or two X groups can be joined together to form a divalent R′ group wherein R′ is a C₁-C₂₀-alkylidene, C₆-C₂₀-arylidene, C₇-C₂₀-alkylarylidene, or C₇-C₂₀-arylalkylidene optionally containing heteroatoms belonging to groups 13-17 of the Periodic Table of the Elements;
R¹, R², R³, R⁴ and R⁵, equal to or different from each other, are hydrogen atoms or C₁-C₄₀ hydrocarbon groups optionally containing one or more heteroatoms belonging to groups 13-17 of the Periodic Table of the Elements;
Z is a divalent radical selected from: CR⁶, S, O, NR⁶, PR⁶, N, P provided that at least one Z is different from CR⁶, preferably provided that only one Z is different from CR⁶; wherein R⁶ equal to or different from each other, are hydrogen atoms or C₁-C₄₀ hydrocarbon groups optionally containing one or more heteroatoms belonging to groups 13-17 of the Periodic Table of the Elements;
n ranges from 3 to 4;
W is a C₆-C₄₀-aryl aryl radical that can be substituted with one or more, linear or branched C₁-C₄₀-alkyl, C₂-C₄₀ alkenyl, C₂-C₄₀ alkynyl radicals.

2. The catalyst system according to claim 1 wherein:
X is a C₇-C₄₀-alkylaryl radical
R¹, R², R³, R⁴ and R⁵ are hydrogen atoms
Z is a divalent radical selected from: CR⁶, S, O, NR⁶, PR⁶, N, P provided that only one
Z is different from CR⁶; and provided that the ring formed by Z is aromatic;
R⁶ equal to or different from each other, are hydrogen atoms or C₁-C₄₀-alkyl, C₆-C₄₀-aryl radicals
W is a phenyl radical substituted in position 2 and 6 by G groups,

3. The catalyst system according to claim 1 having formulas (Ia) or (Ib) 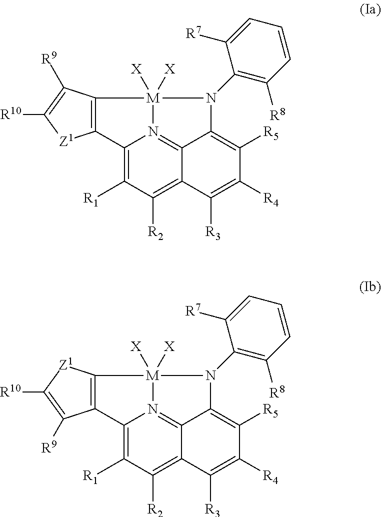
Wherein M, X, R¹, R², R³, R⁴ and R⁵ have the same meaning as in claim 1;
R⁷ and R⁸, equal to or different from each other, are C₁-C₄₀ hydrocarbon groups optionally containing one or more heteroatoms belonging to groups 13-17 of the Periodic Table of the Elements;
R⁹ and R¹⁰, equal to or different from each other, are hydrogen atoms or C₁-C₄₀ hydrocarbon groups optionally containing one or more heteroatoms belonging to groups 13-17 of the Periodic Table of the Elements or R⁹ and R¹⁰ can be joined to form a C₅-C₆ membered ring;
Z¹ is S, O, or NR⁶ wherein R⁶ have the same meaning as in claim 1.

4. The catalyst system according to claim 1 wherein in the compounds of formula (Ia) and (Ib):
R⁷ and R⁸ are linear or branched C₁-C₁₀-alkyl radicals;
R¹⁰ is a C₁-C₁₀-alkyl radical or is joined with R⁹ to form a phenyl ring;
R⁹ is a hydrogen atom or is joined with R¹⁰ to form a phenyl ring;
Z¹ is S or NR⁶ wherein R⁶ is a C₁-C₁₀-alkyl or a C₆-C₂₀-aryl radical.

5. The catalyst system according to claim 1 wherein the activator b) is selected from the group consisting of alumoxanes, boron compounds having Lewis acidity, and mixtures thereof.

6. The catalyst system according to claim 1 further comprising an inert support c).

7. The catalyst system according to claim 1 wherein the inert support c) is silica

8. An organic ligand of formula (II) 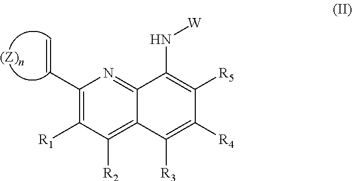
Wherein Z, n, R¹, R², R³, R⁴, R⁵ and W have the same meaning as in claim 1.

9. The organic ligand according to claim 8 having formula (IIa) or (IIb) 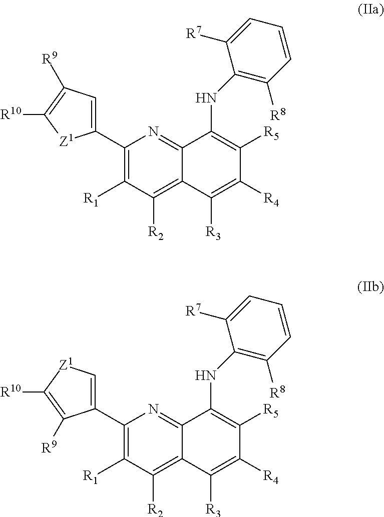
Wherein Z¹, R¹, R², R³, R⁴, R⁵, R⁷, R⁸, R⁹ and R¹⁰ have the same meaning as in claim 1.

10. A process for polymerizing one or more alpha olefins of formula CH₂═CHT wherein T is hydrogen or a C₁-C₂₀ alkyl radical comprising the step of contacting said alpha-olefins of formula CH₂═CHT under polymerization conditions in the presence of the catalyst system of claim 1.

11. The polymerization process according to claim 1 from the polymerization of ethylene and optionally one or more alpha olefins selected from propylene, 1-butene, 1-hexene and 1-octene comprising the step of contacting ethylene and optionally said alpha-olefins under polymerization conditions in the presence of the catalyst system of claim 1.

12. A Group 4 transition metal complex of formula (I) 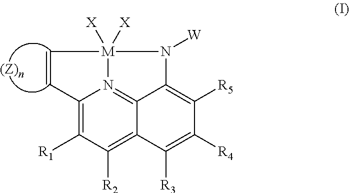
Wherein M, X, Z, n, W, R¹, R², R³, R⁴ and R⁵ have the same meaning as in claim 1;
